(19) United States
(12) Patent Application Publication
(10) Pub. No.: US 2024/0165059 A1
(43) Pub. Date: May 23, 2024

(54) GRANULES CONTAINING AN ACTIVE SUBSTANCE, METHOD FOR PREPARING SAME AND USE THEREOF IN FOOD FOR HUMAN CONSUMPTION OR ANIMAL FEED

(71) Applicant: CAPSULAE, La Rochelle (FR)

(72) Inventors: Carine CHAIGNEAU, Perigny (FR); Robert HUET, La Rochelle (FR); Jean-Eudes VENDEVILLE, Perigny (FR)

(73) Assignee: CAPSULAE, La Rochelle (FR)

(21) Appl. No.: 18/279,916

(22) PCT Filed: Mar. 4, 2022
(86) PCT No.: PCT/EP2022/055609

§ 371 Date: Sep. 1, 2023

(30) Foreign Application Priority Data
Mar. 5, 2021 (FR) FR2102169

(51) Int. Cl. A61K 31/19; A61K 9/16

(52) U.S. Cl. CPC A61K 31/19; A61K 9/1617; A61K 9/1694

(57) ABSTRACT

A granule comprising: sodium butyrate particles, a fatty material matrix comprising fatty acids encapsulating the sodium butyrate particles, the granule preserving its morphology following gastric and enteric digestion simulation in vitro tests, the granule featuring a gastric resistance conferring protection on the sodium butyrate particles in the stomach, and featuring a sustained release of the sodium butyrate particles in the intestinal tract.

### TECHNICAL FIELD

The present invention relates to granules containing an active substance, a preparation method thereof and use thereof in human or animal feed. In particular the invention relates to granules containing sodium butyrate.

### Technical Problem

Compounds derived from butyric acid have many advantageous biological effects, in particular on the digestive system, by stimulating the growth of the intestinal walls and the development of the microorganisms of the intestinal flora. In particular, they selectively feature an antimicrobial effect on some strains of microorganisms of the digestive system. For example, they limit the development of bacteria: Clostridium acetobutylicum, Escherichia Coli, Streptococcus cremoris, Lactococcus lactis and cremoris, and Salmonella strains, while Lactobacillus and Streptococcus bovis strains are less affected by these.

However, like butyric acid, these derived compounds have a strong rancid butter odour which complicates production and storage thereof.

Among these compounds derived from butyric acid, sodium butyrate has the advantage of being in the solid state and stable up to a temperature above 250° C. However, sodium butyrate is sensitive to acidic environments such as those found in the stomach, where it hydrolyzes to form butyric acid which is then volatile or in liquid form facilitating absorption thereof in the stomach. Thus, direct oral absorption of sodium butyrate particles primarily results in dissolution and absorption in the stomach, which limits the uptake of sodium butyrate into the intestinal tract.

The intestinal tract is constituted by the small intestine and the large intestine terminating in the colon. In each of these portions, the environment evolves and differs in particular by the pH and the presence of enzymes. The protection of the sodium butyrate particles should enable dissolution along the intestinal tract and not only a bioavailability at the level of the small intestine. In particular, there is a need for protection enabling an enteric release of sodium butyrate particles in the large intestine, in particular in the colon.

The patent EP 2 352 386 describes a method for the preparation of granules containing sodium butyrate particles in a fatty material matrix wherein calcium sulfate is incorporated into the matrix in order to promote the gastric resistance of the granule.

The patent EP 2 727 472 teaches a method for preparing granules of butyrate particles in a fatty material by extrusion, which then involves coating with a gastric protective layer.

The PCT application WO 2018/033935 describes a process by successive sprays in order to obtain a multilayer granule of sodium butyrate, fatty acid and minerals crossing the gastric barrier of ruminants.

There is a need to protect sodium butyrate particles from the acidic environment of the stomach so that they cross the gastric barrier while preserving their bioavailability along the intestinal tract.

Also, one of the aims of the present invention is to provide granules comprising sodium butyrate particles protected in a fatty material matrix, featuring a gastric resistance conferring thereon protection in the stomach and allowing conferring thereon a sustained release in the intestinal tract.

Another aim of the invention is to provide a population of granules comprising sodium butyrate particles, in the form of stable powders, easily handled and adapted to the application for which said granules are intended.

Another aim of the invention is to provide a method for the preparation of granules comprising sodium butyrate particles, having gastric resistance and appropriate intestinal release properties.

Another aim of the invention consists in providing an animal or human food composition comprising such granules.

### INVENTION

The present invention relates to a granule comprising:
sodium butyrate particles,
a fatty material matrix comprising fatty acids encapsulating said sodium butyrate particles,
said granule preserving its morphology following gastric and enteric digestion simulation in vitro tests,
said granule featuring gastric resistance conferring protection on the sodium butyrate particles in the stomach and featuring a sustained release of the sodium butyrate particles in the intestinal tract.

The Inventors have surprisingly and unexpectedly demonstrated that a granule comprising sodium butyrate particles encapsulated in a fatty material matrix containing fatty acids and obtained under specific preparation conditions preserves its morphology in gastric and enteric digestion media. This results in a structural resistance of the matrix, after digestion in vitro tests simulating the environment of the stomach, the small intestine and the large intestine, this structural resistance allowing for a gastric resistance of the sodium butyrate particles in the stomach and a sustained release of sodium butyrate particles along the intestinal tract.

The gastric resistance and the sustained release are improved compared to granules of the prior art. Thus, a product marketed by Adisseo under the reference ERP80 corresponding to the commercial product Adimix® precision, features a release of sodium butyrate of 85% at the gastric simulation step alone, another granule of encapsulated butyrate marketed by Novation under the trade name of Butirex C4 features a complete release of butyrate in the stomach.

By “gastric digestion”, it should be understood the degradation of ingested products in the stomach by gastric acidity and gastric juices.

The gastric digestion may be simulated in vitro by a solution having a composition close to gastric fluid.

The gastric fluid (or gastric juice) secreted by the stomach contains agents such as hydrochloric acid and some enzymes, such as pepsin, which lyses the proteins.

In general, gastric conditions may be simulated by a solution comprising water maintained at an acid pH comprised between 1 and 4 and a proteolytic enzyme, for example of the pepsin type, said enzyme being present in a content ranging from 0.025 to 2.5% by weight, with respect to the total weight of the solution.

Pepsin degrades the food bolus proteins by hydrolyzing the amino acid peptide bonds.

The used gastric digestion simulation in vitro test is adapted from Boisen's method (Boisen et al., Animal Feed Science Technology 68 (1997), 277-286). This test consists in incubating an amount of granules, for example 1 g, in a solution at pH=2 in the presence of pepsin, for example 25 mg, for 2 hours at 39° C. simulating the gastric environment.

By “enteric digestion”, it should be understood the enteric digestion in the small intestine and that in the large intestine.

By “enteric digestion in the small intestine”, it should be understood the degradation of the ingested products in the environment of the small intestine.

In general, the enteric digestion in the small intestine is simulated in vitro by a solution having a composition close to the fluid present in the small intestine, in particular comprising pancreatin.

Pancreatin is an enzyme derived from the pancreatic juice.

The used in vitro test simulating the enteric digestion in the small intestine is adapted from Boisen's method. This test consists in incubating an amount of granules, for example 1 g, in a solution at pH=6.8 in the presence of pancreatin, for example 100 mg, for 4 hours at 39° C. simulating the enteric environment of the small intestine.

By “enteric digestion in the large intestine”, it should be understood the degradation of the ingested products in the enteric environment of the large intestine.

The enteric digestion in the large intestine is simulated in vitro by a solution having a composition close to the fluid present in the large intestine, in particular comprising lipase. Lipase is a digestive enzyme secreted by the pancreas.

The used in vitro test simulating the enteric digestion in the large intestine is adapted from Boisen's method. This test consists in incubating an amount of granules, for example 1 g, in a solution at pH=7 in the presence of lipase, for example 100 mg, for 18 hours at 39° C. simulating the enteric environment of the large intestine.

By “morphology of the granule”, it should be understood the external shape of the granule.

The Inventors have surprisingly observed that after the digestion tests adapted according to Boisen's method, the granules of the invention preserve their morphology.

The maintenance of the granule morphology indicates that the structure of the fatty material matrix of the granules is preserved for the three tests simulating the gastric and enteric digestions

By “gastric resistance”, it should be understood a resistance to the degradation of granules in the gastric environment. This “gastric resistance” is generally determined by the residual amount of sodium butyrate in the granule after simulation of the gastric passage, for example for two hours in a gastric fluid medium.

By “sustained release”, it should be understood a gradual release of sodium butyrate from its encapsulating fatty material matrix along the digestive tract, generally by digestion or solubilization of the encapsulating fatty material matrix by digestive enzymes, bile salts or microorganisms.

This “sustained release” is generally determined by the amount released from the sodium butyrate by the granules after simulation of the enteric passage, for example for four hours in a fluid simulating the small intestine and for 18 hours in a fluid simulating the large intestine.

The morphological analysis of the granules may be carried out by observation using an optical or electron microscope, preferably using a scanning electron microscope.

According to an advantageous embodiment, the present invention relates to a granule comprising:
sodium butyrate particles,
a fatty material matrix comprising fatty acids encapsulating said sodium butyrate particles,
said granule preserving its morphology following gastric and enteric digestion simulation in vitro tests,
said granule featuring a gastric resistance conferring protection on the sodium butyrate particles in the stomach and featuring a sustained release of sodium butyrate particles in the intestinal tract
and wherein the butyric acid level is lower than 5%, in particular lower than 2%, in particular lower than 1%, in particular lower than 0.5%, preferably lower than 0.1% of the total amount of sodium butyrate.

The butyric acid that could be present in the granule originates from the hydrolysis of sodium butyrate.

According to an advantageous embodiment, the present invention relates to a granule as defined before, wherein the gastric protection rate (TRC1) of the sodium butyrate is higher than or equal to 50%.

According to an advantageous embodiment, the present invention relates to a granule as defined before, wherein the gastric protection rate (TRC1) of the sodium butyrate is higher than or equal to 65%.

According to an advantageous embodiment, the present invention relates to a granule as defined before, wherein the gastric protection rate (TRC1) of the sodium butyrate is higher than or equal to 70%.

By “higher than or equal to 50%”, it should be also understood the following ranges: from 50% to 55%; from 55% to 60%, from 60% to 65%; from 65 to 70%; from 70% to 75%; from 75% to 80%; from 80% to 85%; from 85% to 90%, from 90% to 95% and from 95% to 100%.

By “higher than or equal to 65%”, it should be also understood the following ranges: from 60% to 65%; from 65% to 70%; from 70% to 75%; from 75% to 80%; from 80% to 85%; from 85% to 90%, from 90% to 95% and from 95% to 100%.

By “higher than or equal to 70%”, it should be also understood the following ranges: from 70% to 75%; from 75% to 80%; from 80% to 85%; from 85% to 90%, from 90% to 95% and from 95% to 100%.

By “TRC1 gastric protection rate”, it should be understood the relative amount of sodium butyrate protected from gastric digestion after the above-described in vitro test of incubation in the appropriate gastric environment according to Boisen's method. It corresponds to a percentage of sodium butyrate protected during gastric digestion and which is available for release in the intestinal tract.

The TRC1 gastric protection rate of sodium butyrate is calculated after the above-described in vitro test of incubation in the gastric environment, according to the following formula:

$$TRC 1 = \frac{Qt - Qd}{Qt}$$

wherein Qt represents the initial total amount of sodium butyrate and Qd represents the dissolved amount of sodium butyrate during gastric digestion. The TRC1 gastric protection rate is defined by the ratio between the amount of sodium butyrate retained in the granule and the initial total amount of sodium butyrate in the granule.

The amount of retained butyrate is assessed by subtracting from the total initial amount of sodium butyrate Qt, the amount of sodium butyrate dissolved in solution Qd after the above-described in vitro test of incubation in the gastric environment.

The dissolved amount of sodium butyrate in solution is obtained by quantitative analysis techniques known to those skilled in the art such as GC-MS or colorimetric assays, for example using violet crystal.

For comparison, the products of the prior art such as Adimix® precision, available on the market, lead to a measured a gastric protection rate of 15%. The product disclosed in EP 2 352 386 reports a gastric protection of 61%.

According to an advantageous embodiment, the present invention relates to a granule as defined before, featuring a sustained release of sodium butyrate particles over the entire length of the intestinal tract,
the TRC2 enteric release rate of sodium butyrate in the small intestine being higher than or equal to 25%,
the TRC3 enteric release rate of sodium butyrate in the large intestine being higher than or equal to 50%.

By “higher than or equal to 25%”, it should be also understood the following ranges: from 25% to 30%; from 30% to 35%; from 35% to 40%; from 40% to 45%; from 45% to 50%; from 50% to 55%; from 55% to 60%, from 60% to 65%; from 65% to 70%; from 70% to 75%; from 75% to 80%; from 80% to 85%; from 85% to 90%, from 90% to 95% and from 95% to 100%.

Said TRC2 and TRC3 enteric release rates are analyzed after the enteric digestion in vitro tests adapted according to Boisen's method simulating the above-described enteric environments.

By “TRC2 release rate”, it should be understood the relative amount of sodium butyrate released and dissolved in solution after the above-described in vitro test of incubation in the enteric environment of the small intestine, adapted according to Boisen's method. This TRC2 rate characterizes the released and available amount of sodium butyrate during enteric digestion in the small intestine.

The TRC2 release rate of sodium butyrate is calculated after the above-described in vitro test of incubation in the enteric environment of the small intestine, according to the following formula:

$$TRC 2 = \frac{Qd}{Qt}$$

wherein Qt represents the initial total amount of sodium butyrate and Qd represents the dissolved amount of sodium butyrate during enteric digestion in the small intestine.

It is defined by the ratio between the amount of sodium butyrate released and dissolved in solution of the granule and the initial total amount of sodium butyrate of the granule.

For comparison, the prior art product Adimix® precision allows for a TRC2 enteric release of 87%.

By “TRC3 release rate”, it should be understood the relative amount of sodium butyrate released and dissolved in solution after the above-described in vitro test of incubation in the enteric environment of the large intestine, adapted according to Boisen's method. This TRC3 level characterizes the released and available amount of sodium butyrate during enteric digestion in the large intestine.

The TRC3 release rate of sodium butyrate is calculated after the above-described in vitro test of incubation in the enteric environment of the large intestine. It is defined by the ratio between the amount of sodium butyrate released and dissolved in solution of the granule and the initial total amount of sodium butyrate of the granule.

The amount of sodium butyrate dissolved in solution is obtained by quantitative analysis techniques known to those skilled in the art, such as GC-MS or colorimetric assays.

The sustained release may be characterized by determining TRC2 and TRC3 enteric release rates.

The TRC2 and TRC3 rates enables a comparison of the release capacity of the sodium butyrate particles with the products of the prior art.

A TRC2 rate higher than 25% indicates a release of 25% of the initial total amount of sodium butyrate from the granule into the enteric environment of the small intestine.

A TRC3 rate higher than 50% indicates a release of 50% of the initial total amount of sodium butyrate from the granule into the enteric environment of the large intestine.

According to an advantageous embodiment, the present invention relates to a granule as defined before, wherein the TRC3 release rate of sodium butyrate in the large intestine is higher than or equal to 60%.

According to an advantageous embodiment, the present invention relates to a granule as defined before, wherein the TRC3 release rate of sodium butyrate in the large intestine is higher than or equal to 70%.

For comparison, the prior art product Adimix® precision allows for a TRC3 enteric release of 79%.

According to an advantageous embodiment, the present invention relates to a granule as defined before, wherein the gastric protection rate (TRC1) of sodium butyrate is higher than or equal to 50%, in particular 65%, preferably 70%,
and/or featuring a sustained release of sodium butyrate particles over the entire length of the intestinal tract,
the TRC2 enteric release rate of sodium butyrate in the small intestine being higher than or equal to 25%,
the TRC3 enteric release rate of sodium butyrate in the large intestine being higher than or equal to 50% respectively, in particular higher than or equal to 65%, preferably higher than or equal to 70%.

According to an advantageous embodiment, the invention relates to a granule comprising:
sodium butyrate particles,
a fatty material matrix comprising fatty acids encapsulating said sodium butyrate particles,
said granule preserving its morphology following gastric and enteric digestion simulation in vitro tests,
said granule featuring a gastric resistance conferring protection on the sodium butyrate particles in the stomach and featuring a sustained release of sodium butyrate particles in the intestinal tract and
wherein the gastric protection rate (TRC1) of sodium butyrate is higher than or equal to 50%, in particular 65%, preferably 70%, the TRC1 gastric protection rate being the relative amount of sodium butyrate protected from gastric digestion after the in vitro test of incubation in the gastric environment adapted according to Boisen's method
and/or
featuring a sustained release of the sodium butyrate particles over the entire length of the intestinal tract,
wherein
the TRC2 enteric release rate of sodium butyrate in the small intestine is higher than or equal to 25%, the TRC2 release rate being the relative amount of sodium butyrate released and dissolved in solution after the in vitro test of incubation in the enteric environment of the small intestine, adapted according to Boisen's method,
the TRC3 enteric release rate of sodium butyrate in the large intestine is higher than or equal to respectively 50%, in particular higher than or equal to 65%, preferably higher than or equal to 70%, the “TRC3 release rate” being the relative amount of sodium butyrate released and dissolved in solution after the in vitro test of incubation in the enteric environment of the large intestine, adapted according to Boisen's method.

According to an advantageous embodiment, the invention relates to a granule comprising:
sodium butyrate particles,
a fatty material matrix comprising fatty acids encapsulating said sodium butyrate particles,
said granule preserving its morphology following gastric and enteric digestion simulation in vitro tests,
said granule featuring a gastric resistance conferring protection on the sodium butyrate particles in the stomach and featuring a sustained release of sodium butyrate particles in the intestinal tract and
wherein the gastric protection rate (TRC1) of sodium butyrate is higher than or equal to 50%, in particular 65%, of preferably 70%, the TRC1 gastric protection rate being the relative amount of sodium butyrate protected from gastric digestion after the in vitro test of incubation in the gastric environment adapted according to Boisen's method and/or
featuring a sustained release sodium butyrate particles over the entire length of the intestinal tract, wherein:
the TRC2 enteric release rate of sodium butyrate in the small intestine is higher than or equal to 25%, the TRC2 release rate being the relative amount of butyrate of sodium released and dissolved in solution after the in vitro test of incubation in the enteric environment of the small intestine, adapted according to Boisen's method,
the TRC3 enteric release rate of sodium butyrate in the large intestine is respectively higher than or equal to 50%, in particular higher than or equal to 65%, preferably higher than or equal to 70%, the “TRC3 release rate” being the relative amount of sodium butyrate released and dissolved in solution after the in vitro test of incubation in the enteric environment of the large intestine, adapted according to Boisen's method and
wherein the butyric acid level is lower than 5%, in particular lower than 2%, in particular lower than 1%, in particular lower than 0.5%, preferably lower than 0.1% of the total amount of sodium butyrate.

According to an advantageous embodiment, the present invention relates to a granule as defined before, wherein said fatty material comprises long-chain fatty acids containing more than 12 carbon atoms, and in particular from 12 to 22 carbon atoms, preferably containing 12, 14, 16, 18, 20 and 22 carbon atoms, still more preferably 16 and 18 carbon atoms.

By “C16”, it should be understood a fatty acid containing a chain of 16 carbon atoms.

Similarly, by “C18”, it should be understood a fatty acid containing a chain of 18 carbon atoms.

According to an advantageous embodiment, the present invention relates to a granule as defined before, wherein said fatty material comprises long-chain fatty acids containing 16 and 18 carbon atoms, in particular at a content higher than or equal to 70% of the total weight of the fatty material.

By “higher than or equal to 70%”, it should be also understood the following ranges: from 70% to 75%; from 75% to 80%; from 80% to 85%; from 85% to 90%, from 90% to 95% and from 95% to 100%.

According to an advantageous embodiment, the present invention relates to a granule as defined before, wherein the C16/C18 weight ratio of the fatty acids is from 0.7 to 1.7.

By “from 0.7 to 1.7”, it should be also understood the following ranges: from 0.7 to 0.8; from 0.8 to 0.9; from 0.9 to 1.0; from 1.0 to 1.1; from 1.1 to 1.2; from 1.2 to 1.3; from 1.3 to 1.4; from 1.4 to 1.5; from 1.5 to 1.6; from 1.6 to 1.7.

According to an advantageous embodiment, the present invention relates to a granule wherein the fatty material comprises:
from 40% to 65% C16 fatty acid
and from 30% to 60% C18 fatty acid,
wherein the sum of the percentages of C16 and C18 fatty acids is less than 1.

By “from 40% to 65%”, it should be also understood the following ranges: from 40% to 45%; from 45% to 50%; from 50% to 55%; from 55% to 60% and from 60% to 65%.

By “from 30% to 60%”, it should be also understood the following ranges: from 30% to 35%; from 35 to 40%; 40% to 45%; from 45% to 50%; from 50% to 55% and from 55% to 60%.

According to an advantageous embodiment, the present invention relates to a granule wherein the C16:C18 weight ratio of the fatty acids is from 1.0 to 1.7; in particular 1.1 or 1.6 or 1.7.

According to an advantageous embodiment, the present invention relates to a granule wherein the fatty material comprises:
from 50% to 60% C16 fatty acid
and from 30% to 45% of C18 fatty acid,
wherein the sum of the percentages of C16 and C18 fatty acids is less than 1.

Preferably, the fatty material of the granule comprises 57% C16 fatty acids and 36% C18 fatty acids.

Preferably, the fatty material of the granule comprises 59% C16 fatty acids and 35% C18 fatty acids.

Preferably, the fatty material of the granule comprises 55% C16 fatty acids and 41% C18 fatty acids.

According to an advantageous embodiment, the present invention relates to a granule of the invention, wherein the C16:C18 weight ratio of the fatty acids is from 0.7 to 1.0; in particular 0.8 or 0.9.

According to an advantageous embodiment, the present invention relates to a granule wherein the fatty material comprises:
from 40% to 50% C16 fatty acid
and from 50% to 60% of C18 fatty acid,
wherein the sum of the percentages of C16 and C18 fatty acid is less than 1.

Preferably, the fatty material of the granule comprises 44% C16 fatty acids and 54% C18 fatty acids.

Preferably, the fatty material of the granule comprises 46% C16 fatty acids and 52% C18 fatty acids.

According to an advantageous embodiment, the present invention relates to a granule as defined before, wherein said fatty material comprises long-chain fatty acids containing more than 12 carbon atoms, and in particular from 12 to 22 carbon atoms, in in particular long-chain fatty acids containing 16 and 18 carbon atoms, said C16 and C18 fatty acids being in particular at a content higher than or equal to 70% of the total weight of the fatty material, the C16:C18 weight ratio of the fatty acids being in particular from 0.7 to 1.7.

According to an advantageous embodiment, the present invention relates to a granule as defined before, wherein the morphology is spherical.

By “spherical morphology”, it should be understood in the context of the present invention, a regular morphology with an aspect ratio close to 1.

By “aspect ratio”, it should be understood in the context of the present invention, the ratio between the size of the axis of largest dimension, so-called main axis, and the size of the axis of smallest dimension, so-called secondary axis, of the granule. A particle is considered as spherical from a principal axis to secondary axis ratio lower than 1.1.

The analysis may be performed by shape recognition type tools, by image analysis, for example with the ELLIX software from Microvision Instruments, version 6.0.2. It enables a measurement of particle size, circularity and orientation and may be used to characterize sphericity. This operating software is coupled to a camera for image capture.

The sphericity of the granule confers thereon powder compacting properties enabling easier storage and transport.

The sphericity of the granule also allows minimizing the surface of exchange of the granules with the outside allowing for a limitation of the degradations.

In particular, advantageously, the spherical morphology of the granules of the invention allows for a lower exchange surface than that of a granule with an elongated or ovoid morphology of the same volume. Thus, the spherical granule of the invention allows minimizing, by limiting the exchange surface, the degradation by gastric or enteric juices of the stomach and intestine and preserving its structural integrity allowing for a sustained release along the intestinal tract.

According to an advantageous embodiment, the present invention relates to a granule as defined before, wherein the matrix does not comprise any mineral. The absence of minerals in the matrix is advantageous from a cost perspective.

According to an advantageous embodiment, the present invention relates to a granule as defined before, wherein the matrix comprises one mineral or several minerals.

By “mineral”, it should be understood an inorganic substance.

The minerals may have several functions. They may be used in a process as a buffer. They may also be incorporated to generate or improve the properties of the granules.

According to an advantageous embodiment, the present invention relates to a granule as defined before, wherein the matrix comprises a mineral percentage of 2% to 10% of the total weight of the granule.

By “from 2% to 10%”, it should be also understood the following ranges: from 2% to 3%; from 3% to 4%; from 4% to 5%; from 5% to 6%; from 6% to 7%; from 7% to 8%; from 8% to 9%; from 9 to 10%.

According to an advantageous embodiment, the present invention relates to a granule as defined before, wherein said mineral is selected from among calcium carbonate, tricalcium phosphate (TCP), calcium sulfate, calcium silicate, magnesium sulfate, magnesium carbonate, aluminum phosphate, cobalt carbonate, zinc carbonate and mixtures thereof.

Surprisingly, in contrast with the patent EP 2 352 386, the particles obtained with the present invention containing calcium sulfate feature a gastric protection (TRC1) of 71 and 73% in comparison with a protection of 15% for the product Adimix® precision.

According to an advantageous embodiment, the present invention relates to a granule as defined before, wherein said mineral is tricalcium phosphate (TCP), in particular at a content of 2% to 10% of the total weight of the granule.

According to an advantageous embodiment, the present invention relates to a granule as defined before, said granule comprising or not comprising calcium sulfate, in particular said granule comprising calcium sulfate.

According to an advantageous embodiment, the present invention relates to a granule as defined before, wherein said mineral is calcium sulfate, in particular at a content of 2% to 10% of the total weight of the granule.

According to an advantageous embodiment, the present invention relates to a granule as defined before, wherein the matrix comprises one mineral or several minerals, in particular at a mineral percentage of 2% to 10% of the total weight of the granule, preferably said mineral being selected from among calcium carbonate, tricalcium phosphate, calcium sulfate, calcium silicate, magnesium sulfate, magnesium carbonate, aluminum phosphate, cobalt carbonate, zinc carbonate and mixture thereof, in particular tricalcium phosphate and calcium sulfate.

According to an advantageous embodiment, the present invention relates to a granule as defined before, wherein said granule comprises:
from 40% to 80% by weight of fatty material,
and from 20% to 60% by weight of sodium butyrate.

By “from 40% to 80%”, it should be also understood the following ranges: from 40% to 50%; from 50% to 60%; from 60% to 70%; from 70% to 80%.

By “from 20% to 60%”, it should be also understood the following ranges: from 20% to 30%; from 30% to 40%; from 40% to 50%; from 50% to 60%.

According to an advantageous embodiment, the present invention relates to a granule as defined before, wherein said granule comprises:
from 50% to 70% by weight of fatty material,
and from 30% to 50% by weight of sodium butyrate.

By “from 30% to 70%”, it should be also understood the following ranges: from 30% to 40%; from 40% to 50%; from 50% to 60%; from 60% to 70%.

By “from 30% to 50%”, it should be also understood the following ranges: from 30% to 35%; from 35% to 40%; from 40% to 45%; from 45% to 50%.

According to an advantageous embodiment, the present invention relates to a granule as defined before, wherein said granule comprises 80% by weight of fatty material and 20% by weight of sodium butyrate.

According to an advantageous embodiment, the present invention relates to a granule as defined before, wherein said granule comprises 70% by weight of fatty material, 10% by weight of minerals and 20% by weight of sodium butyrate.

According to an advantageous embodiment, the present invention relates to a granule as defined before, wherein said granule comprises 70% by weight of fatty material and 30% by weight of sodium butyrate.

According to an advantageous embodiment, the present invention relates to a granule as defined before, wherein said granule comprises 60% by weight of fatty material, 10% by weight of minerals and 30% by weight of sodium butyrate.

According to an advantageous embodiment, the present invention relates to a granule as defined before, wherein said granule comprises 50% by weight of fatty material and 50% by weight of sodium butyrate.

According to an advantageous embodiment, the present invention relates to a granule as defined before, wherein the fatty material is selected from among the group consisting of hydrogenated palm oil, hydrogenated sunflower oil, hydrogenated rapeseed oil, beeswax, candelilla wax, carnauba wax, palm stearin, stearic acid or a mixture thereof.

According to an advantageous embodiment, the present invention relates to a granule as defined before, wherein the fatty material is hydrogenated palm oil.

Hydrogenated palm oil may be commercially available and is supplied for example by Mosselman s.a. (Belgium) or ADM-SIO (France).

According to an advantageous embodiment, the present invention relates to a granule as defined before, wherein said granule comprises:
from 40% to 80% by weight of fatty material, preferably from 50% to 70%,
and from 20% to 60% by weight of sodium butyrate, preferably from 30% to 50%,
and wherein the fatty material is selected in particular from the group consisting of hydrogenated palm oil, hydrogenated sunflower oil, hydrogenated rapeseed oil, beeswax, candelilla wax, carnauba, palm stearin, stearic acid or mixtures thereof.

According to an advantageous embodiment, the present invention relates to a granule as defined before, wherein said matrix comprises air bubbles inside the granule.

According to an advantageous embodiment, the present invention relates to a granule as defined before, said granule containing no emulsifiers.

By “emulsifier”, it should be understood an additive allowing creating a stable and homogeneous emulsion. Emulsifiers to avoid are, for example, polyethylene glycols (PEG), polysorbates (Tween 20 or 80), sunflower, soya or rapeseed lecithins, mono and diglycerides of fatty acids.

According to an advantageous embodiment, the present invention relates to a granule as defined before, with no unpleasant odor.

By “unpleasant odor”, it should be understood the release of malodorous molecules into the air.

By “with no unpleasant odor”, it should be understood a release, in particular of butyric acid, at low levels that do not inconvenience the user during the production, handling or storage of the granules.

According to an advantageous embodiment, the present invention relates to a granule as defined before, wherein said granule does not comprise an additional outer protective coating layer. The granule according to the invention has the advantage of not having to incorporate a gastric protective layer or a protective layer against degradation thereof by the air. Indeed, butyric acid, resulting in a strong rancid odor which complicates handling and storage thereof, is not released by the granules of the invention, given their structure. The stability of the granules also has an advantage in terms of safety. Indeed, the REACH regulation (EC No. 1907/2006) recommends an exposure threshold for workers of 36.8 mg/m³. With regards to the storage of products, it should be noted that butyric acid has a lower explosive limit of 2% by volume, i.e. 2,000 ppm. According to an advantageous embodiment, the present invention relates to a granule as defined before, wherein the level of free butyric acid is lower than 5%, in particular lower than 2%, in particular lower than 1%, in particular lower than 0.5%, preferably lower than 0.1%.

According to an advantageous embodiment, the present invention relates to a granule as defined before, wherein the level of free butyric acid is zero.

By “free butyric acid level”, it should be understood the butyric acid level in the free form present in the granule. This rate may be determined by extracting a granule after grinding in an organic solvent such as hexane wherein butyric acid is soluble and wherein sodium butyrate is insoluble. The amount of butyric acid extracted into the organic solvent may be determined by analytical techniques such as gas chromatography. The level of free butyric acid is defined by the ratio between this molar amount extracted from butyric acid and the initial total molar amount of sodium butyrate and butyric acid from the granule.

By “lower than 5%”, it should be understood the following ranges lower than 5%, 4%, 3%, 2%, 1%.

By “lower than 0.5%”, it should be understood the following ranges lower than 0.5%; 0.4%; 0.3%; 0.2%; 0.1%.

By “lower than 0.1%”, it should be understood the following ranges lower than 0.1%; 0.09%; 0.08%; 0.07%; at 0.06%; 0.05%; 0.04%; 0.03%; 0.02%, 0.01%, 0.001%.

By “zero level”, it should be understood a level that cannot be detected by analysis by gas-phase chromatography.

According to an advantageous embodiment, the present invention relates to a granule as defined before, wherein the degree of esterification is lower than 15%, preferably lower than 10%, in particular lower than 1.0%

By “esterification level”, it should be understood the level of butyric acid bound to fatty acids or fatty acid triglycerides. This level is defined as the ratio between the molar amount of bound butyric acid and the total initial molar amount of butyrate.

This level may be determined by inverse titration by ion chromatography after grinding the granule and water extraction. Indeed, ion chromatography allows determining the total amount of sodium butyrate and butyric acid, without distinguishing between the acid form and the basic form, that being due to the extraction in aqueous phase. In the presence of fatty acid or fatty acid triglyceride, sodium butyrate may form butyric acid esters by esterification. These esters do not have the same spectrum in ion chromatography as sodium butyrate and butyric acid.

By “lower than 15%”, it should be understood the following ranges lower than 15%, 14%, 13%, 12%, 11%, 10%, 9%, 8%, 7%, 6%, 5%, 4%, 3%, 2%, 1%.

By “lower than 1.0%”, it should be understood the following ranges lower than 0.9%; 0.8%; 0.7%: 0.6%; 0.5%; 0.4%, 0.3%; 0.2%; 0.1%.

The analysis of the Admix® precision product indicates the presence of an esterification level higher than 16%. The presence of butyric acid ester in Admix® precision is confirmed by phase-change analyses in a basic medium enabling the butyrate bound to fatty acids to be saponified and released.

The butyric acid form, namely in the form of free butyric acid, in the form of sodium butyrate or in the form of an ester wherein it is bound to fatty acids or fatty acid triglycerides, is one factors that determine its bioavailability in the intestinal tract.

The invention also relates to a population of granules, said granules being as defined before.

According to an advantageous embodiment, the present invention relates to a population of granules as defined before, wherein the particle size of the granules varies from 200 μm to 1.5 mm, preferably from 400 μm to 1,000 μm, preferably 600 to 800 μm. According to an advantageous embodiment, the present invention relates to a population of granules as defined before, wherein the mean particle size Dv(0.5) is from 600 to 800 μm, in particular 630 μm.

The measurement of the mean particle size Dv(0.5) of the Adimix® precision product is about 990 μm.

By average particle size Dv(0.5), it should be understood the average particle size diameter, such that 50% of the granules of said population having a diameter larger than said average diameter and 50% of the particles of said composition having a diameter smaller than said average diameter.

As regards ovoid particles, the average diameter takes consideration of the width and the length of the particles.

The average particle size diameter may be measured by laser diffraction or by sieving.

According to an advantageous embodiment, the present invention relates to a population of granules as defined before, wherein the average size of the sodium butyrate particles varies from 50 to 1,200 μm, in particular from 100 to 800 μm, preferably from 200 at 300 μm.

According to an advantageous embodiment, the present invention relates to a population of granules as defined before, wherein the sodium butyrate particles are in the form of compacted grains.

By “compacted grains”, it should be understood grains formed by compacting fine powder of sodium butyrate using compactor-granulator type equipment, for example Alexanderwerk WP120.

According to an advantageous embodiment, the present invention relates to a population of granules as defined before, wherein the SPAN value of the granules is lower than or equal to 1.0; in particular lower than or equal to 0.8; preferably lower than or equal to 0.5; said SPAN value being calculated according to the following formula:

$$SPAN = \frac{D ( 90 \% ) - D ( 10 \% )}{D ( 50 \% )}$$

wherein D(90%), D(50%) and D(10%) represent the diameters for which respectively 90%, 50% and 10% of the population of granules has a diameter smaller than this value.

The SPAN value of a population of granules is a dispersion index of the size of the granules in the population.

In the case of a SPAN value lower than 0.5, said population is considered as monodisperse.

According to an advantageous embodiment, the present invention relates to a population of granules as defined before, wherein the population of granules forming a powder has a flow index from 4 to 7 [Flodex™ index], in particular 4 or 5.

The Flodex® method (Dow-Lepetit) measures the fluidity (or ability to flow) of a powder. A sample is placed in a smooth cylinder having circular orifices with different sizes (ranging from 4 to 34) in the bottom. The orifices are sealed during filling. Once the total amount of powder has been introduced, the orifices are opened. A powder with good fluidity flows through a small section orifice, whereas a powder with poor fluidity requires a large section orifice to come out of the cylinder. The Flodex™ flow index is equal to the diameter, in millimeters, of the smallest orifice through which the powder has fallen three times in a row.

A flow index of 4 to 7 is considered as indicating an excellent flow.

A flow index of 8 to 12 is considered as indicating a good flow.

A flow index of 14 to 18 is considered as indicating an average flow.

A flow index of 20 to 28 is considered as indicating a fair flow.

A flow index of 28 to 34 is considered as indicating a poor flow.

According to an advantageous embodiment, the present invention relates to a population of granules as defined before, wherein the apparent density of the population of granules is from 0.45 to 0.65 g/cm³.

According to an advantageous embodiment, the present invention relates to a population of granules as defined before, wherein the packed density of the population of granules is from 0.50 to 0.71 g/cm³.

The measurement of the apparent density and of the packed density is carried out according to the AFNOR NF V 04-344 standard.

According to an advantageous embodiment, the present invention relates to a population of granules as defined before, wherein the moisture pick-up value is from 3% to 10% after 24 hours, in particular from 5 to 10%.

The moisture pick-up is measured in a sealed desiccator, maintained at a relative humidity of 75% by a saturated NaCl solution, and maintained at 25° C. 2 g of powder are weighed in a pre-calibrated cup.

The cup is kept in this humidity-controlled atmosphere for a period of 24 hours. The moisture pick-up is measured every hour for 5 hours and then at 24 hours.

The moisture pick-up is measured in % moisture pick-up relative to the initial moisture.

The moisture pick-up reflects the hygroscopic nature of a powder, or the ability of a powder to capture water, and to dissolve afterwards. By extension, this method indirectly indicates the rate of solubilization of the compounds of a powder.

According to an advantageous embodiment, the present invention relates to a population of granules as defined before,
wherein the grain size of the granules varies from 200 μm to 1.5 mm, preferably from 400 μm to 1,000 μm, preferably 600 to 800 μm, and in particular wherein the size of the sodium butyrate particles varies from 50 at 1,200 μm, in particular from 200 to 300 μm, or
wherein the SPAN value of the granules is lower than or equal to 1; in particular lower than or equal to 0.8; preferably lower than or equal to 0.5; said SPAN value being calculated according to the following formula:

$$SPAN = \frac{D ( 90 \% ) - D ( 10 \% )}{D ( 50 \% )}$$

wherein D(90%), D(50%) and D(10%) represent the diameters for which respectively 90%, 50% and 10% of the population of granules has a diameter smaller than this value,
   or wherein the population of granules forming a powder has a flow index of 4 to 7 [Flodex™ index], in particular 4 or 5,
   or wherein the moisture pick-up value is from 3 to 10%, in particular from 5 to 10%.

According to an advantageous embodiment, the present invention relates to a population of granules as defined before, wherein at least 90% by weight of the granules has a spherical morphology.

According to an advantageous embodiment, the present invention relates to a population of granules as defined before, which is odorless.

According to an advantageous embodiment, the present invention relates to a population of granules as defined before, wherein said granule does not comprise an additional outer protective coating layer. The population of granules according to the invention has the advantage of stability with regards to degradation in the air, without the use of a protective layer, thereby enabling handling and storage without inconvenience by the odor.

The invention also relates to a method for preparing a population of granules comprising:
sodium butyrate particles,
a fatty material matrix comprising fatty acids, encapsulating said sodium butyrate particles,
said granule preserving its morphology following gastric and enteric digestion simulation in vitro tests,
said granule featuring a gastric resistance conferring protection on the sodium butyrate particles in the stomach and featuring a sustained release of sodium butyrate particles in the intestinal tract,
said method comprising:
a step of preparing a mixture of sodium butyrate particles in the solid state in the liquid of said fatty material in the molten state to obtain a suspension
a step of forming granules from said suspension, possibly subsequently solidified in the form of a dispersion of particles in the crystallized fatty material,
wherein the viscosity of the suspension consisting of the mixture of the sodium butyrate particles in the molten liquid of the fatty material before the aforementioned step of forming the granules is lower than 8,000 mPa·s, preferably lower than 5,000 mPa·s, more preferably lower than 2,500 mPa·s, in particular from 10 to 8,000 mPa·s.

The viscosity is measured using a Brookfield digital DV-E viscometer as follows: the viscosity is analyzed by introducing an amount of 10 ml of product to be analyzed into the thermostatically-controlled measurement chamber of the Brookfield digital DV-E viscometer. The viscometer is configured with a coaxial cylinder in the thermostatically-controlled measuring chamber, using a reference S31 rotating spindle. The viscosity measurement temperature is 85° C., maintained by a thermostatically-controlled water bath. The viscosity is determined for a rotational speed of the spindle of 10 rpm.

The Inventors have observed an increase in the viscosity of the liquid fatty material during the introduction of the sodium butyrate particles into the liquid fatty material during the step of preparing the suspension of the method. At a viscosity lower than 8,000 mPa·s, it is possible to obtain granules according to the invention having the properties of gastric protection and sustained enteric release reported before in the description of the invention.

By “from 10 to 8,000 mPa·s”, it should be also understood the following ranges: from 10 to 100 mPa·s; from 10 to 200 mPa·s; from 10 to 300 mPa·s; from 10 to 500 mPa·s; from 10 to 1,000 mPa·s; from 10 to 1,500 mPa·s; from 10 to 2,000 mPa·s; from 10 to 2,500 mPa·s; from 10 to 3,000 mPa·s; from 10 to 4,000 mPa·s; from 10 to 5,000 mPa·s from 10 to 6,000 mPa·s; from 10 to 7,000 mPa·s; from 10 to 8,000 mPa·s.

The viscosity of the suspension constituted by the mixture of the sodium butyrate particles in the molten liquid of the fatty material before the aforementioned step of forming the granules may be lower than 8,000 mPa·s, 7,000 mPa·s, 6,000 mPa·s, 5,000 mPa·s, 4,000 mPa·s, 3,000 mPa·s, 2,500 mPa·s, 2,000 mPa·s, 1,500 mPa·s, 1,000 mPa·s, 900 mPa·s, 800 mPa·s, 700 mPa·s, 600 mPa·s, 500 mPa·s, 400 mPa·s, 300 mPa·s, 250 mPa·s, 200 mPa·s.

In a preferred embodiment of the method of the invention, the mixture is maintained in the form of the aforementioned suspension for a time period shorter than or equal to 15 minutes, in particular for a time period from 2 seconds to 10 minutes.

According to an advantageous embodiment, the present invention relates to a method wherein the temperature of the fatty material in the molten state in liquid form is at a temperature higher by 5° C. to 30° C. than the melting point of the fatty material.

By “from 5° C. to 30° C.”, it should be also understood the following ranges: from 5° C. to 10° C., from 10° C. to 15° C., from 15° C. to 20° C., from 20° C. to 25° C., from 25° C. to 30° C.

According to an advantageous embodiment, the present invention relates to a method wherein the temperature of the fatty material in the molten state in liquid form is at a temperature from 50° C. to 120° C., in particular from 65° C. to 110° C., preferably from 65° C. to 95° C.

By “From 50° C. to 120° C.”, it should be also understood the following ranges: from 50° C. to 60° C., from 60° C. to 70° C., from 70° C. to 80° C., from 80° C. at 90° C., from 90° C. to 100° C., from 100° C. to 110° C., from 110° C. to 120° C.

According to an advantageous embodiment, the present invention relates to a method as defined before, comprising:
a step of preparing a mixture of a powder of sodium butyrate particles in a fatty material in the molten state in liquid form, to obtain a suspension
a step of forming drops from said suspension
a step of cooling the aforementioned drops into granules enabling the solidification of the fatty material forming a matrix encapsulating said sodium butyrate particles.

According to an advantageous embodiment, the present invention relates to a method as defined before, comprising:
a step of preparing a mixture of a powder of sodium butyrate particles in a fatty material in the molten state in liquid form, by introducing said powder into said fatty material in the molten state in liquid form by appropriate means, in particular using a mixer, to obtain a suspension,
a step of forming drops by spraying the aforementioned suspension,
a step of cooling said drops in a cooling chamber at a temperature of −20° C. to 30° C., in particular under an air stream.

By “from −20° C. to 30° C.”, it should be also understood the following ranges: from −20° C. to −10° C., from −10° C. to 0°, from 0° C. to 10° C., from 10° C. to 20° C. and from 20° C. to 30° C.

According to an advantageous embodiment, said step of preparing a mixture is carried out in a device such as static or dynamic mixers, in particular mixers, extruders, and mixers with no internal parts, such as ultrasonic mixers.

According to an advantageous embodiment, said step of preparing a mixture is carried out in a static mixer.

According to an advantageous embodiment, said step of preparing a mixture is carried out in a dynamic mixer.

According to an advantageous embodiment, said step of preparing a mixture is carried out in an extruder.

According to an advantageous embodiment, the present invention relates to a method as defined before, comprising:
a step of preparing a mixture of a powder of sodium butyrate particles and fatty material in powder form, followed by raising the temperature of said mixture to melt said fatty material containing the sodium butyrate particles to obtain a suspension,
a step of forming the drops of the aforementioned suspension,
a step of cooling said drops in a cooling chamber at a temperature from −20° C. to 30° C., in particular under an air stream.

According to an advantageous embodiment, said step of preparing the mixture is carried out in an extruder.

According to an advantageous embodiment, said step of preparing the mixture is carried out in a thermostatically-controlled tank with a small size with a capacity of 0.5 to 5 liters.

According to an advantageous embodiment, said step of preparing the mixture is carried out in a dynamic mixer comprising an inlet into a reactor for the fatty material previously melted in the liquid state and an inlet for the addition of the butyrate powder.

According to an advantageous embodiment, the present invention relates to a method as defined before, comprising:
a step of preparing a mixture of a powder of sodium butyrate particles and fatty material in powder form, followed by raising the temperature of said mixture to melt said fatty material containing the sodium butyrate particles to obtain a suspension,
a step of cooling said suspension to obtain a dispersion of particles in the crystallized fatty material,
a step of forming granules from said dispersion,
a step of cooling said granules.

According to an advantageous embodiment, said step of preparing a mixture from powder is carried out in an extruder.

According to an advantageous embodiment, the present invention relates to a method as defined before,
comprising:
a step of preparing a mixture of a powder of sodium butyrate particles in a fatty material in the molten state in liquid form to obtain a suspension,
a step of forming drops from said suspension,
a step of cooling said drops into granules enabling the solidification of the fatty material forming a matrix encapsulating said sodium butyrate particles;
or comprising:
a step of preparing a mixture of a powder of sodium butyrate particles in a fatty material in the molten state in liquid form, by introducing said powder into said fatty material in the molten state in liquid form by appropriate means, in particular using a mixer, to obtain a suspension,
a step of forming drops by spraying the aforementioned suspension,
a step of cooling said drops in a cooling chamber at a temperature of −20° C. to 30° C., in particular under an air stream:
or comprising:
a step of preparing a mixture of a powder of sodium butyrate particles and fatty material in powder form, followed by raising the temperature of said mixture to melt said fatty material containing the sodium butyrate particles, to obtain a suspension
a step of forming the drops of the aforementioned suspension,
a step of cooling said drops in a cooling chamber at a temperature of −20° C. to 30° C., in particular under an air stream:
or comprising:
a step of preparing a mixture of a powder of sodium butyrate particles and fatty material in powder form, followed by raising the temperature of said mixture to melt said fatty material containing the sodium butyrate particles to obtain a suspension,
a step of cooling said suspension to obtain a dispersion of particles in the crystallized fatty material,
a step of forming granules from said dispersion.

According to an advantageous embodiment, the present invention relates to a method as defined before, wherein said fatty material comprises long-chain fatty acids containing more than 12 carbon atoms, and in particular from 12 to 22 carbon atoms.

According to an advantageous embodiment, the present invention relates to a method as defined before, wherein said fatty material comprises long-chain fatty acids containing 16 and 18 carbon atoms, in particular at a content higher than or equal to 70% of the total fatty material weight.

According to an advantageous embodiment, the present invention relates to a method as defined before, wherein the C16:C18 weight ratio of the fatty acids is from 0.7 to 1.7.

According to an advantageous embodiment, the present invention relates to a method as defined before, wherein said fatty material comprises long-chain fatty acids containing 16 and 18 carbon atoms, in particular at a content higher than or equal to 70% of the total weight of the fatty material, preferably the C16:C18 weight ratio of the fatty acids is from 0.7 to 1.7.

According to an advantageous embodiment, the present invention relates to a method as defined before, wherein the fatty material comprises:
from 40% to 65% C16 fatty acid
and from 30% to 60% C18 fatty acid,
wherein the sum of the percentages of C16 and C18 fatty acids is less than 1.

According to an advantageous embodiment, the present invention relates to a method as defined before, wherein the C16:C18 weight ratio of the fatty acids is from 1.0 to 1.7; in particular 1.1 or 1.6 or 1.7.

According to an advantageous embodiment, the present invention relates to a method as defined before, wherein the fatty material comprises:
from 50% to 60% C16 fatty acid
and from 30% to 45% of C18 fatty acid,
wherein the sum of the percentages of C16 and C18 fatty acids is less than 1.

Preferably, the fatty material of the method as defined before comprises 57% C16 fatty acids and 36% C18 fatty acids.

Preferably, the fatty material of the method as defined before comprises 59% C16 fatty acids and 35% C18 fatty acids.

Preferably, the fatty material of the method as defined before comprises 55% C16 fatty acids and 41% C18 fatty acid.

According to an advantageous embodiment, the present invention relates to a method as defined before, wherein the C16:C18 weight ratio of the fatty acids is from 0.7 to 1.0; in particular 0.8 or 0.9.

According to an advantageous embodiment, the present invention relates to a method as defined before, wherein the fatty material comprises:
from 40% to 50% C16 fatty acid
and from 50% to 60% C18 fatty acid,
wherein the sum of the percentages of C16 and C18 fatty acid is less than 1.

Preferably, the fatty material of the method as defined before comprises 44% C16 fatty acids and 54% C18 fatty acids.

Preferably, the fatty material of the method as defined before comprises 46% C16 fatty acids and 52% C18 fatty acids.

According to an advantageous embodiment, the present invention relates to a method as defined before, wherein said fatty material comprises long-chain fatty acids containing 16 and 18 carbon atoms, in particular at a content higher than or equal to 70% of the total weight of the fatty material, preferably the C16:C18 weight ratio of the fatty acids is from 0.7 to 1.7.

According to an advantageous embodiment, the present invention relates to a method as defined before, wherein the fatty material matrix does not comprise any mineral.

According to an advantageous embodiment, the present invention relates to a method as defined before, wherein the fatty material matrix does not comprise any mineral, and wherein the viscosity of the suspension is lower than 5,000 mPa·s, in particular from 10 to 5,000 mPa·s.

According to an advantageous embodiment, the present invention relates to a method as defined before, wherein the fatty material matrix comprises at least one mineral.

According to an advantageous embodiment, the present invention relates to a method as defined before, wherein the fatty material comprises a mineral and wherein the viscosity of the suspension formed is lower than 8,000 mPa·s in particular from 10 to 8,000 mPa·s.

According to an advantageous embodiment, the present invention relates to a method as defined before, wherein the fatty material matrix comprises at least one mineral in an amount of 2% to 10% of the total weight of the granule.

According to an advantageous embodiment, the present invention relates to a method as defined before, wherein said mineral is calcium carbonate, in particular at a content from 2 to 10% of the total weight of the granule, preferably 5% or 10%.

According to an advantageous embodiment, the present invention relates to a method as defined before, wherein said mineral is tricalcium phosphate (tricalcium phosphate), in particular at a content of 2% to 10% of the total weight of the granule.

According to an advantageous embodiment, the present invention relates to a method as defined before, wherein said mineral is tricalcium phosphate and has a content of 5%.

According to an advantageous embodiment, the present invention relates to a method as defined before, wherein said mineral is calcium sulfate, in particular at a content of 2 to 10% of the total weight of the granule.

According to an advantageous embodiment, the present invention relates to a method as defined before, wherein the calcium sulfate is at a content of 2 to 10% of the total weight of the granule and wherein the C16:C18 weight ratio of the fatty acids is 1.0 to 1.7; in particular 1.1 or 1.6 or 1.7.

According to an advantageous embodiment, the present invention relates to a method as defined before, wherein the calcium sulfate is present at a content of 2% and the calcium carbonate is at a content of 5% of the total weight of the granule, and wherein the fatty material matrix comprises 57% C16 fatty acids and 36% C18 fatty acids.

According to an advantageous embodiment, the present invention relates to a method as defined before, wherein the fatty material matrix comprises at least one mineral in an amount of 2% to 10% of the total weight of the granule, said mineral being in particular tricalcium phosphate or calcium sulfate.

According to an advantageous embodiment, the present invention relates to a method as defined before, wherein the viscosity is adjusted by known methods, in particular by adding inorganic or organic additives.

According to an advantageous embodiment, the present invention relates to a method as defined before, wherein said method is implemented in the absence of emulsifiers.

By “emulsifier”, it should be understood an additive allowing creating a stable and homogeneous emulsion. The emulsifiers to be avoided are, for example, polyethylene glycols (PEG), polysorbates (Tween 20 or 80), sunflower, soya or rapeseed lecithins, mono or diglycerides.

According to an advantageous embodiment, the present invention relates to a method as defined before, which does not comprise an additional step of coating the granules.

The invention also relates to a suspension of liquid fatty material containing sodium butyrate particles having a viscosity lower than 8,000 mPa·s.

According to an advantageous embodiment, the present invention relates to a suspension described hereinabove wherein the fatty material is hydrogenated palm oil.

The invention also relates to a population of granules which can be obtained according to a method as defined before.

Another object of the invention relates to an animal or human feed composition comprising granules or a population of granules as defined before.

### DESCRIPTION OF THE FIGURES

FIG. 1 is an optical microscopy image of the PR1G1F granules obtained according to the invention.

FIG. 2 is a set of scanning electron microscopy SEM images of a product of the prior art Adimix® precision (1st line) and of two products prepared according to the invention PR1G1F and PCaG1R1 in the 2nd and 3rd lines.

The figures in the first line 2 a), b) and c) are images of the granules of the prior art, Adimix® precision, having as a composition 30% sodium butyrate, 63% fatty material, 5% carbonate calcium and 2% calcium sulfate.

The figures in the second line 2 d), e) and f) are those of the granules of the PR1G1F product prepared according to the invention comprising 30% sodium butyrate and 70% fatty material (hydrogenated palm oil) without the addition of minerals.

The figures in the third line 2 g), h) and i) are images of the granules of the PCaG1R1 product prepared according to the invention comprising 30% sodium butyrate and 60% fatty material (hydrogenated palm oil) with a 10% calcium carbonate content.

The figures in the first column 2 a), d) and g) show the initial morphology of the granules before the in vitro gastric or enteric digestion tests.

The figures in the second column 2 b), e) and h) show the morphology of the granules after the in vitro gastric digestion test at pH 2 in the presence of pepsin, for 2 hours at 39° C.

The figures in the third column 2 c), f) and i) show the morphology of the granules after the in vitro enteric digestion test in the presence of lipase at pH 7 for 18 h at 39° C.

The scale bar at the bottom to the right of the scanning electron microscopy images represents 400 μm for the images a), b), c), d), e), f), g) and h) and 600 μm for the image i).

For the 3 analyzed products, the morphology is preserved after the in vitro gastric digestion test.

For the granules prepared according to the invention, the products PR1G1F and PCaG1R1, although cracks are observed on the granules, the initial spherical morphology is preserved after the enteric digestion test with lipase. In the case of the Adimin® precision product, the coating matrix degrades after enteric incubation leading to a loss of the initial morphology of the granules.

Thus, the granules prepared according to the invention PR1G1F and PCaG1R1 preserve the structure of the coating matrix both in the gastric and enteric environment.

FIG. 3 represents the optical microscopy images used for morphological analysis by the Ellix image analysis software. FIG. 3a) is the overview of the granules used for the analysis of the PR1G1F product, prepared according to the invention comprising 30% sodium butyrate and 70% fatty material (hydrogenated palm oil) without the addition of minerals, without minerals; FIG. 3b) that one used for the analysis of the product PCaR1G1F, prepared according to the invention comprising 30% sodium butyrate and 65% fatty material with a content of 5% tricalcium phosphate; FIG. 3c) that one used for the analysis of the product Adimix® precision.

FIG. 4 is a table representing the morphology of the granules using optical microscopy images compared to the measured viscosity of the suspension before the granule formation step.

### EXAMPLES

### Example 1

First, in a container, we weigh at room temperature (between 20 and 25° C.): 1.2 kg of fine grade powdered sodium butyrate (90% minimum smaller than 200 μm measured by a sieving method) and 2.8 kg of hydrogenated palm oil (supplier MOSSELMAN) in the form of solid particles. The contents of this container are poured into a tumbler mixer to stir the mixture for 5 minutes. A homogeneous mixture of the 2 powders is obtained, at room temperature (between 20 and 25° C.).

This mixture is introduced into a filling hopper and feeds via a powder doser at a rate of 7 kg/h a thermoregulated extruder equipped with a thermostatically-controlled wall. The extruder heating setpoint is set at 90° C.

Under the effect of pressure and calories, the hydrogenated palm oil becomes liquid, which sets the sodium butyrate in suspension in the liquid fatty material,

The configuration of the extruder is made with a screw length of 15 cm, the rotation is 35 rotations per minute (rpm), finally the product flow rate is 7 kg/h.

The obtained suspension comes out of the extruder throughout the outlet orifice, in liquid form.

The suspension is sent by gravity into a thermoregulated turbine at 70° C., located at the top of an atomization tower, enabling the formation of drops of 700 μm (+/−200 μm) in an atomization chamber with a counter-current of cold air at a regulated temperature between 15° C. and 20° C. enabling the solidification of the suspension in the form of spherical particles.

The particles then correspond to a dispersion of sodium butyrate in a fatty material matrix. The obtained particles have a spherical shape, with a size from 600 μm to 1 mm.

### Example 2

The use of a thermoregulated extruder allows carrying out the steps of mixing, blending and shaping granules in one single piece of equipment in a temperature range from 20 to 10° C.

5 kg of hydrogenated palm oil (supplier MOSSELMAN) are prepared in a tank at a temperature of 70° C. until the fatty material is obtained in the molten state.

Fine grade powdered sodium butyrate (90% minimum under 200 μm measured by a sieving method) is poured into the filling hopper of the 1st section of the extruder. A powder doser feeds the extruder with sodium butyrate at a flow rate of 1.2 kg/h.

The liquid hydrogenated palm oil is injected at a flow rate of 2.8 kg/h into the 2nd section of the extruder via an injector located 10 cm from the filling hopper where the sodium butyrate is dosed.

The configuration of the extruder is made with a screw length of 15 cm, the rotation is 35 rotations per minute, finally the product flow rate is 4 kg/h.

The passage through the extrusion screws at a temperature between 70 and 85° C. allows obtaining a suspension of butyrate in the liquid fatty material in a homogeneous manner before coming out through the extrusion die.

The product is cooled to 25° C. at the outlet of the extruder, and comes out of the die in the form of cylinders of solid product.

A rotating disc with blades located 3 mm from the die cuts the stems of products that come out into regular particles. The particles have a length of 750 μm+/−200 μm and a diameter of 700 μm.

Afterwards, these particles are spheronized in a piece of equipment regulated at 50° C. and rotating at 500 rpm so as to make these particles spherical.

The particles then correspond to a dispersion of sodium butyrate in a fatty material matrix

### Example 3

10 kg of hydrogenated palm oil (supplier MOSSELMAN) are incorporated into a melter, equipped with a double jacket with a heating setpoint of 90° C., until complete meltdown of the fatty material is obtained, which is therefore in the liquid state.

The melted fatty material is drawn from the bottom of the tank using a Watson Marlow type peristaltic pump equipped with thermostatically-controlled heating cords, set at a flow rate of 7 kg/h.

The pump feeds a thermostatically-controlled mixing tank, with a capacity of 5 liters, allowing for a capacity of 2.5 to 3 kg of suspension, having a double jacket maintained at 90° C., and equipped with a bladed propeller stirring of the KA RW20 type.

This same thermostatically-controlled mixing tank is fed with fine grade powdered sodium butyrate (90% minimum smaller than 200 μm measured by a sieving method), thanks to a powder dispenser at a flow rate of 3 kg/h. The mixing tank allows evenly distributing the sodium butyrate in the liquid fatty material in less than 20 seconds. A liquid suspension is then obtained.

The liquid suspension is drawn at the outlet of the thermostatically-controlled tank using a pump at a flow rate of 10 kg/h and transferred to the spray nozzle.

The spraying of the fatty material/butyrate suspension is carried out by a nozzle of the two-fluid type with inner mixing (Spraying System), in an enclosure at room temperature (between 20 and 25° C.).

A matrix product is obtained where the butyrate particles are inside a fatty material matrix.

The obtained product is a powder composed of spherical granules constituting a dispersion of sodium butyrate in the fatty material matrix.

The composition of the final mixture is then 70% hydrogenated palm oil and 30% sodium butyrate.

### Example 4

4,200 g of hydrogenated palm oil (supplier MOSSELMAN) are incorporated into a melter, equipped with a double jacket with a heating setpoint of 90° C., until complete meltdown of the fatty material is obtained, which is therefore in the liquid state.

The melted fatty material is drawn from the bottom of the tank, using a Watson Marlow type peristaltic pump equipped with thermostatically-controlled heating cords, set at a flow rate of 700 g/h, the pump feeds a thermostatically-controlled reactor. This same thermostatically-controlled reactor is fed with fine grade powdered sodium butyrate (90% minimum smaller than 200 μm measured by a sieving method), thanks to a powder doser at a flow rate of 300 g/h. The homogeneous liquid suspension of sodium butyrate in the liquid fatty material is obtained in less than 30 seconds.

This suspension is transferred at the outlet of the thermostatically-controlled reactor via a thermostatically-controlled pipe to a spraying device known to those skilled in the art (turbine, rotating disc, two-fluid spray nozzle).

The spraying of the fatty material/butyrate suspension is carried out in an enclosure at room temperature (between 20 and 25° C.) which allows freezing the suspension drops.

The obtained product is a powder composed of spherical granules with an average particle size of 400 microns constituting a dispersion of sodium butyrate in the fatty material matrix.

### Example 5

In this example, we proceed as in Example 4.
4,200 g of hydrogenated palm oil (supplier MOSSELMAN) are placed in a melter, equipped with a double jacket with a heating setpoint of 90° C., until complete meltdown of the fatty material is obtained, which is therefore in the liquid state.
600 g of fine grade powdered sodium butyrate (90% minimum smaller than 200 μm measured by a sieving method) are mixed with 100 g of tricalcium phosphate to obtain a homogeneous powder mixture containing 85.7% sodium butyrate and 14, 3% tricalcium phosphate. This mixture is poured into a powder dispenser.

The melted fatty material is drawn from the bottom of the tank, using a Watson Marlow type peristaltic pump equipped with thermostatically-controlled heating cords, set at a flow rate of 650 g/h, the pump feeds a thermostatically-controlled reactor.

This same thermostatically-controlled reactor is fed with the mixture of butyrate powder and tricalcium phosphate by a powder dispenser at a flow rate of 350 g/h.

The homogeneous liquid suspension of the sodium butyrate and mineral powders in the liquid fatty material is obtained in less than 30 seconds.

This suspension is transferred at the outlet of the thermostatically-controlled reactor through a thermostatically-controlled pipe to a rotating disc.

The spraying of the fatty material/butyrate and mineral suspension is carried out in an enclosure at room temperature (between 20 and 25° C.) which allows freezing the suspension drops.

The obtained product is a powder composed of spherical granules constituting a dispersion of sodium butyrate in the fatty material matrix.

The composition of the final mixture is then 65% hydrogenated palm oil, 5% tricalcium phosphate, 30% sodium butyrate.

### Example 6

The same operation as that of Example 5 is carried out, by implementing 10% calcium carbonate with respect to the total formula, 60% hydrogenated palm oil and 30% sodium butyrate.

The calcium carbonate is mixed in the proper proportions with the sodium butyrate powder.

The melted fatty material is drawn from the bottom of the tank, using a Watson Marlow type peristaltic pump equipped with thermostatically-controlled heating cords, set at a flow rate of 650 g/h, the pump feeds a thermostatically-controlled reactor.

This same thermostatically-controlled reactor is fed with the mixture of butyrate powder and tricalcium phosphate by a powder dispenser at a flow rate of 350 g/h.

The homogeneous liquid suspension of the sodium butyrate and mineral powders in the liquid fatty material is obtained in less than 30 seconds.

This suspension is transferred at the outlet of the thermostatically-controlled reactor through a thermostatically-controlled pipe to a rotating disc.

The spraying of the fatty material/butyrate suspension is carried out in an enclosure at room temperature (between 20 and 25° C.) which allows freezing the suspension drops.

The obtained product is a powder composed of spherical granules constituting a dispersion of sodium butyrate and calcium carbonate in the fatty material matrix.

The composition of the final mixture is then 60% hydrogenated palm oil, 10% calcium carbonate, and 30% sodium butyrate.

### Example 7

Finally, the same implementation as Example 5 is performed, with 5% calcium carbonate, 2% calcium sulfate, 30% sodium butyrate and 63% hydrogenated palm oil.

The calcium salts are mixed in the proper proportions with the sodium butyrate powder.

The melted fatty material is drawn from the bottom of the tank, using a Watson Marlow type peristaltic pump equipped with thermostatically-controlled heating cords, set at a flow rate of 650 g/h, the pump feeds a thermostatically-controlled reactor.

This same thermostatically-controlled reactor is fed with the mixture of butyrate powder and tricalcium phosphate by a powder dispenser at a flow rate of 350 g/h.

The homogeneous liquid suspension of the sodium butyrate and mineral powders in the liquid fatty material is obtained in less than 30 seconds.

This suspension is transferred at the outlet of the thermostatically-controlled reactor through a thermostatically-controlled pipe to a rotating disc.

The spraying of the fatty material/butyrate suspension is carried out in an enclosure at room temperature (between 20 and 25° C.) which allows freezing the suspension drops.

The obtained product is a powder composed of spherical granules constituting a dispersion of sodium butyrate and calcium carbonate in the fatty material matrix.

The composition of the final mixture is then 63% hydrogenated palm oil, 5% calcium carbonate, 2% calcium sulfate, and 30% sodium butyrate.

### Example 8

15,000 kg of hydrogenated palm oil (supplier SIO) are incorporated into a melter, equipped with a double jacket with a heating temperature of 7000, until complete meltdown of the fatty material is obtained, which is therefore in the liquid state.

500 kg of fine grade powdered sodium butyrate (90% minimum less than 200 μm measured by a sieving method) are transferred into a powder doser.

The melted fatty material is drawn from the bottom of the tank and transferred via a double jacket line thermostated at 80° C. using a volumetric pump, at a flow rate of 390 kg/h. The volumetric pump feeds the thermostatically-controlled reactor.

This same thermostatically-controlled reactor is fed with sodium butyrate powder by a powder doser at a rate of 167 kg/h.

The homogeneous liquid suspension of the sodium butyrate powders in the liquid fatty material is obtained in less than 30 seconds.

This suspension is transferred at the outlet of the thermostated reactor via a pipe thermostated at 75° C. to a two-fluid spray nozzle with inner mixing of the Spraying System type. Granules having a Dv(0.50) of 1 mm are obtained.

The fatty material/butyrate suspension is sprayed via a nozzle, at a flow rate of 557 kg/h, into an atomization chamber with a counter-current of cold air at a regulated temperature between 15 and 20° C. enabling the solidification of the suspension in the form of spherical particles. The obtained solidified particles then correspond to a dispersion of sodium butyrate in a solid fatty material matrix.

The spraying lasted 45 minutes, without interruption.

The obtained powder is composed of spherical particles, the particle size of which, measured by laser granulometry, is characterized by a median Dv(0.50) of 658 μm.

### Example 9: Analysis of the Protection and Release Rates

The assessment of the TRC1 protection and TRC2 and TRC3 release rates has been carried out according to the above-described method. The digestions, gastric and enteric, are carried out as follows.

### In Vitro Gastric Digestion for the Assessment of TRC1

Gastric digestion is simulated by introducing 1 g+/−0.1 mg of granules into an Erlenmeyer flask containing 25 ml of a phosphate buffer solution (pH 6, 0.1 M) and 10 ml HCl (0.2 M). The solution is brought to pH=2 using an HCl or NaOH solution at 1M. Then, 1 ml of a pepsin solution (25 mg/ml), prepared from pepsin from the gastric mucosa porcine (SIGMA ref P-7000, 250 U/mg solid), is added. The solution is incubated at 39° C. for 2 hours. Afterwards, the solution is filtered through a pleated filter to recover the granules which can be observed under a scanning electron microscope in environmental mode, according to the method described in E. Conforto et al. (“An optimized methodology to analyze biopolymer capsules by environmental scanning electron microscopy”, Materials Science and Engineering: C, Volume 47, Feb. 1, 2015, Pages 357-366).

The filtrate is recovered in a 100 ml graduated flask containing 10 ml of 2-methylhexanoic acid, and the butyric acid is assayed according to the standard assay method for volatile fatty acids by gas phase chromatography.

### In Vitro Enteric Digestion in the Small Intestine for the Assessment of TRC2

Enteric digestion is simulated by introducing 1 g+/−0.1 mg of granules into an Erlenmeyer flask containing 25 ml of a phosphate buffer solution (pH 6, 0.1 M) and 10 mL HCl (0.2 M). The solution is brought to pH=6.8 using an HCl or NaOH solution at 1M. Then, 1 mL of a pancreatin solution (100 mg/ml), prepared from pancreatin from porcine pancreas (SIGMA ref P-7545), are introduced into the mixture. The solution is incubated at 39° C. for 4 hours. Afterwards, the solution is filtered to recover the granules which can be observed under a scanning electron microscope in environmental mode, according to the method described in E. Conforto et al. (“An optimized methodology to analyze biopolymer capsules by environmental scanning electron microscopy”, Materials Science and Engineering: C, Volume 47. Feb. 1, 2015, Pages 357-366).

The filtrate is recovered in a 100 ml graduated flask containing 10 ml of 2-methylhexanoic acid, and the butyric acid is assayed according to the standard assay method for volatile fatty acids by gas phase chromatography.

### In Vitro Enteric Digestion in the Large Intestine for the Assessment of TRC3

Enteric digestion is simulated by introducing 1 g+/−0.1 mg of granules into a solution containing 25 ml of a phosphate buffer solution (pH 6, 0.1 M) and 10 mL HCl (0.2 M). The solution is brought to pH=7 using an HCl or NaOH solution at 1M. Then, 100 mg of lipase, derived from porcine pancreas lipase (SIGMA ref. L3126), are introduced into the mixture. The solution is incubated at 39° C. for 18 hours. Afterwards, the solution is filtered to recover the granules which can be observed under a scanning electron microscope in environmental mode, according to the method described in E. Conforto et al. (“An optimized methodology to analyze biopolymer capsules by environmental scanning electron microscopy” Materials Science and Engineering: C, Volume 47, Feb. 1, 2015, Pages 357-366).

The filtrate is recovered in a 100 ml graduated flask containing 10 ml of 2-methylhexanoic acid, and the butyric acid is assayed according to the standard assay method for volatile fatty acids by gas phase chromatography.

Table 1 below reports the values of the gastric protection rates TRC1 and the enteric release rates TRC2 and TRC3 for the granules prepared according to the invention containing the same content (Buty) of sodium butyrate (30%) but at different contents of minerals (from 0 to 10%) and for a reference product from the prior art Adimix® precision. The fatty material (MG) of the matrix for all of the analyzed products is hydrogenated palm oil.

The product referenced PR1G1F without minerals has been prepared according to Example 4. The PCaR1G1F product, containing 5% tricalcium phosphate, has been prepared according to Example 5. The product referenced PCaR1G2F, containing 5% calcium carbonate and 2% calcium sulfate, has been prepared according to Example 7. The product referenced PCaR1 G1, containing 10% calcium carbonate, has been prepared according to Example 6.

TABLE 1
Gastric protection rate TRC1, small intestine enteric release
rate TRC2, large intestine enteric release rate TRC3

| | Composition | | | |
|---|---|---|---|---|
| Reference | Buty/MG/CaCO₃/CaSO₄/Ca₃(PO₄)₂ | TRC1 | TRC2 | TRC3 |
| Adimix ®precision | 30/63/5/2/0 | 15% | 87% | 79% |
| PR1G1F | 30/70/0/0/0 | 79% | 28% | 83% |
| PCaR1G1F | 30/65/0/0/5 | 77% | 28% | 83% |
| PCaR1G2F | 30/63/5/2/0 | 71% | 37% | 93% |
| PCaR1G1 | 30/60/10/0/0 | 66% | 34% | 88% |

The products according to the invention (PR1G1F, PCaR1G1F, PCaR1G2F and PCaR1G1) all have a gastric protection rate TRC1 higher than 65%, i.e. 3 to 4 times higher than the product of the prior art, Adimix® precision.

The TRC2 release rate of the products of the invention is between 25% and 40%, i.e. lower than that of the prior art, Adimix® precision.

The TRC3 release rate of the products of the invention is higher than 80%, i.e. higher than that of the prior art, Adimix® precision.

The TRC2 and TRC2 values show a release kinetic different from that of the prior art, Adimix® precision. In particular, the products of the invention are released later on in the enteric tract, in particular primarily in the enteric environment of the large intestine.

It should be noted that the product of the invention without minerals (PR1G1F) has the highest TRC1 gastric protection rate value at 79%.

It is also observed that although of the same composition with Adimix® precision, the protection and release rate values of PCaR1G2F are different.

### Example 10: Granulometric and Morphological Analysis

a. Morphology of the Granules

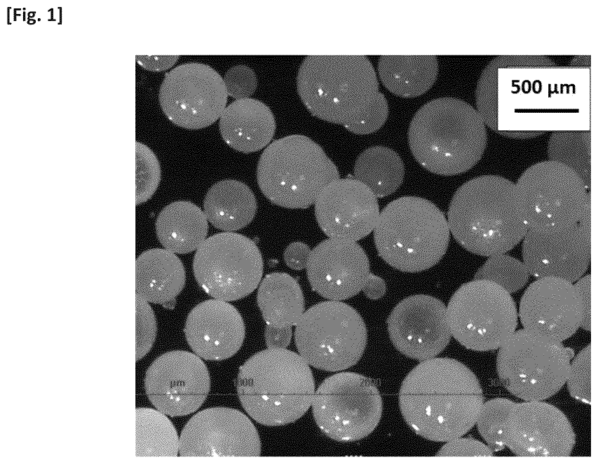

The morphology of the PR1G1F granules, prepared according to the invention comprising 30% sodium butyrate and 70% fatty material (hydrogenated palm oil) without the addition of minerals, is observed using an optical microscope. FIG. 1 shows individualized spherical particles. The monodispersity in size of the powder is confirmed by laser particle size analysis which reveals an average particle size value Dv(0.5) of 630 μm and a SPAN value of 0.638.

For comparison, Adimix® precision granules have an average grain size Dv(0.5) of 990 μm and a SPAN value of 1.450.

b. Morphology Before and After Digestions

The initial morphology of the PR1G1F and PCaR1G1 granules and that after gastric and enteric digestion in vitro are observed by scanning electron microscopy in environmental mode, according to the method described in E. Conforto et al. (“An optimized methodology to analyze biopolymer capsules by environmental scanning electron microscopy”, Materials Science and Engineering: C, Volume 47, February 1st, 2015, Pages 357-366).

### Gastric Digestion In Vitro

Gastric digestion is simulated by introducing 1 g+/−0.1 mg of granules into an Erlenmeyer flask containing 25 ml of a phosphate buffer solution (pH 6, 0.1 M) and 10 ml HCl (0.2 M). The solution is brought to pH=2 using a HCl or NaOH solution at 1M. Then, 1 ml of a pepsin solution (25 mg/ml), prepared from pepsin from porcine gastric mucosa (SIGMA ref P-7000, 250 U/mg solid), is added. The solution is incubated at 39° C. for 2 hours. Afterwards, the solution is filtered through a pleated filter to recover the granules which can be observed under a scanning electron microscope in environmental mode.

Enteric Digestion with Lipase In Vitro

Enteric digestion is simulated by introducing 1 g+/−0.1 mg of granules into a solution containing 25 ml of a phosphate buffer solution (pH 6, 0.1 M) and 10 ml HCl (0.2 M). The solution is brought to pH=7 using an HCl or NaOH solution at 1M. Then, 100 mg of lipase, derived from porcine pancreas lipase (SIGMA ref. L3126), are introduced into the mixture. The solution is incubated at 39° C. for 18 hours.

Afterwards, the solution is filtered to recover the granules which can be observed under a scanning electron microscope in environmental mode.

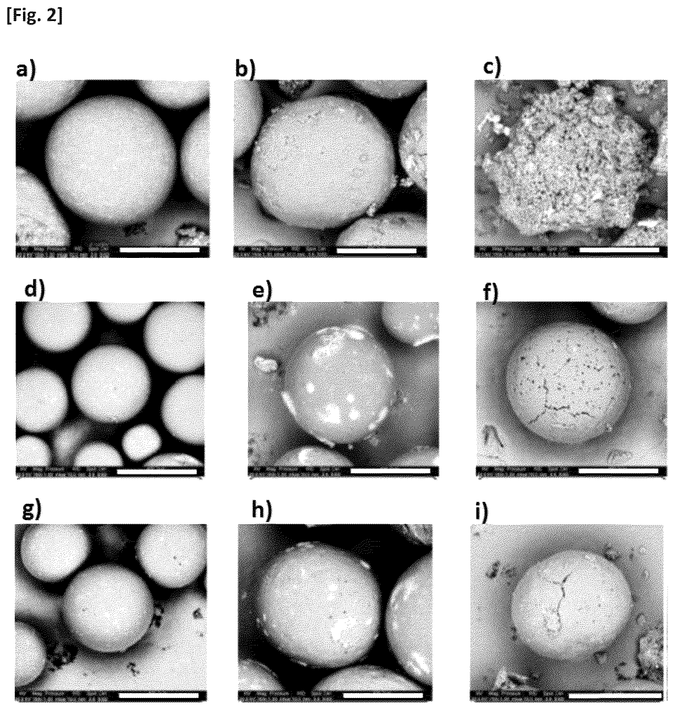

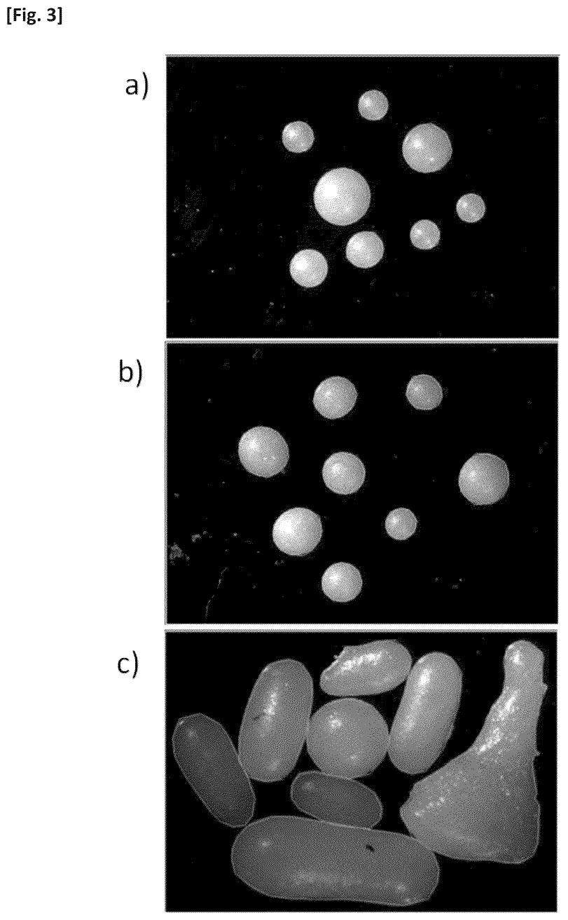

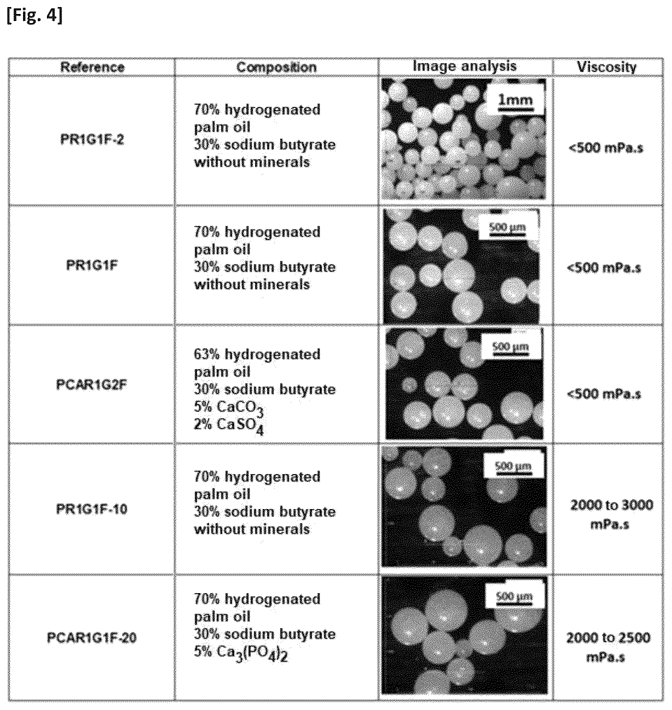

FIG. 2 shows the morphology of the granules of a product of the prior art Adimix® precision (1st line) and of two products prepared according to the invention PR1G1F and PCaR1 G1 in the 2nd and 3rd lines.

The products of the invention PR1G1F and PCaR1G1 preserve their morphology after gastric digestion as well as after gastric digestion.

c. Sphericity Analysis

The analysis of the sphericity of the granules of PR1G1F, PCaR1G1F and Adimix® precision is performed by shape recognition type tools by image analysis with the ELLIX software from Microvision Instruments, version 6.0.2.

The granules are surrounded then their size, their circularity and their orientation are analyzed by the software which gives an index of sphericity by the ratio of the axes of the particles. FIG. 3 shows the images used for morphological analysis by the ELLIX software.

Table 2 represents the data provided by the software with reference to FIG. 3a), image of the granules of the PR1G1F product and the ratio between length and width, aspect ratio index.

TABLE 2
Image analysis data of the granules of FIG. 3a).
PR1G1F

| | Surface | | | Surface | Perimeter | |
|---|---|---|---|---|---|---|
| | of the | | | of the | of the | Length/ |
| | model | Length | Width | object | object | Width |
| Object | [μm²] | [μm] | [μm] | [μm²] | [μm] | ratio |
| 1 | 9,109.2 | 108.90 | 106.60 | 9,109.2 | 342.44 | 1.02 |
| 2 | 5,551.3 | 84.68 | 83.74 | 5,551.3 | 269.23 | 1.01 |
| 3 | 8,911.6 | 109.61 | 103.69 | 8,911.6 | 338.65 | 1.06 |
| 4 | 5,184.5 | 81.94 | 80.70 | 5,184.5 | 258.75 | 1.02 |
| 5 | 21,012.0 | 164.21 | 163.00 | 21,012.0 | 528.75 | 1.01 |
| 6 | 15,591.7 | 141.28 | 140.69 | 15,591.7 | 447.12 | 1.00 |
| 7 | 5,567.9 | 86.94 | 81.78 | 5,567.9 | 268.91 | 1.06 |
| 8 | 6,430.0 | 92.40 | 88.84 | 6,430.0 | 288.24 | 1.04 |

Table 3 represents the data provided by the software with reference to FIG. 3b), image of the granules of the PCaR1G1F product and the ratio between length and width, aspect ratio index.

TABLE 3
Image analysis data of the granules of FIG. 3b).
PCaR1G1F

| | Surface | | | Surface | Perimeter | |
|---|---|---|---|---|---|---|
| | of the | | | of the | of the | Length/ |
| | model | Length | Width | object | object | Width |
| Object | [μm²] | [μm] | [μm] | [μm²] | [μm] | ratio |
| 1 | 11,652.7 | 128.00 | 116.05 | 11,652.7 | 387.41 | 1.10 |
| 2 | 16,506.2 | 150.79 | 139.50 | 16,506.2 | 460.94 | 1.08 |
| 3 | 15,460.7 | 146.14 | 134.87 | 15,460.7 | 446.71 | 1.08 |
| 4 | 10,514.3 | 117.62 | 113.96 | 10,514.3 | 368.02 | 1.03 |
| 5 | 6,418.1 | 91.96 | 88.99 | 6,418.1 | 288.58 | 1.03 |
| 6 | 11,638.4 | 125.60 | 118.08 | 11,638.4 | 386.88 | 1.06 |
| 7 | 17,258.7 | 152.53 | 144.29 | 17,258.7 | 471.53 | 1.06 |
| 8 | 8,249.5 | 107.18 | 98.36 | 8,249.5 | 328.67 | 1.09 |

Table 4 represents the data provided by the software with reference to FIG. 3c), images of the granules of Adimix® precision and the ratio between length and width, aspect ratio index.

TABLE 4
Image analysis data of the granules of FIG. 3c).
Adimix ® precision

| | Surface | | | Surface | Perimeter | |
|---|---|---|---|---|---|---|
| | of the | | | of the | of the | Length/ |
| | model | Length | Width | object | object | Width |
| Object | [μm²] | [μm] | [μm] | [μm²] | [μm] | ratio |
| 1 | 903,702.3 | 1,508.68 | 766.05 | 903,702.3 | 3,692.09 | 1.97 |
| 2 | 510,058.0 | 1,098.37 | 597.25 | 510,058.0 | 2,776.75 | 1.84 |
| 3 | 695,900.9 | 954.20 | 929.24 | 695,900.9 | 2,992.57 | 1.03 |
| 4 | 811,790.9 | 1,503.40 | 691.96 | 811,790.9 | 3,577.47 | 2.17 |
| 5 | 2,339,010.0 | 2,748.39 | 1,374.35 | 2,339,010.0 | 7,372.53 | 2.00 |
| 6 | 497,227.0 | 1,123.20 | 565.84 | 497,227.0 | 2,751.74 | 1.99 |
| 7 | 769,145.8 | 1,468.10 | 671.71 | 769,145.8 | 3,494.59 | 2.19 |
| 8 | 1,919,890.0 | 2,565.08 | 967.66 | 1,919,890.0 | 5,798.09 | 2.65 |

In the case of the PR1G1F and PCaR1G1F granules of the invention, the ratio of the length and width values calculated by the software is lower than or equal to 1.1. This confirms that the granules of the invention are spherical.

In the case of granules from Adimix® precision, the ratio of the length and width values calculated by the software is higher than 1.1 except for one granule, corresponding to the spherical granule at the center of the image in the FIG. 3c). The aspect ratio of the analyzed granules of Adimix® precision confirms the observed visual assessment indicating that most granules do not feature a spherical morphology.

### Example 11: Moisture Pick-Up Analysis

This method is used to determine the water content that a powder picks up over time. This water content is important to provide information on the stability of this powder.

The moisture pick-up is measured in a sealed desiccator, maintained at a relative humidity of 75% by a saturated NaCl solution, and maintained at 25° C.

Between 2 g to 4 g of granule powder are weighed in a pre-calibrated cup.

The cup is kept in this humidity-controlled atmosphere for a period of 24 hours. The moisture pick-up is measured every hour for 5 hours and then at 24 hours.

The moisture pick-up is measured in % moisture pick-up relative to the initial moisture.

Table 5 below reports the moisture pick-up values over time for PR1G1F, a product without minerals in the composition of the fatty material matrix.

TABLE 5
PR1G1F moisture pick-up
PR1G1F

| Time (h) | Mass (g) | % moisture pick-up |
|---|---|---|
| 0 | 2.279 | 0% |
| 1 | 2.284 | 0.22% |
| 2 | 2.285 | 0.26% |
| 4 | 2.289 | 0.44% |
| 5 | 2.296 | 0.75% |
| 24 | 2.483 | 8.95% |

Table 6 below reports the moisture pick-up values over time for PCaR1 G1F, a product of the invention containing 5% tricalcium phosphate (TCP).

TABLE 6
PCaR1G1F moisture pick-up
PCaR1G1F

| Time (h) | Mass (g) | % moisture pick-up |
|---|---|---|
| 0 | 2.194 | 0% |
| 1 | 2.198 | 0.18% |
| 2 | 2.200 | 0.27% |
| 4 | 2.204 | 0.46% |
| 5 | 2.208 | 0.64% |
| 24 | 2.390 | 8.93% |

Table 7 below shows the moisture pick-up values over time for the prior art product Adimix® precision.

TABLE 7
Adimix ®precision moisture pick-up
Adimix ® Precision

| Time (h) | Mass (g) | % moisture pick-up |
|---|---|---|
| 0 | 2.242 | 0% |
| 1 | 2.251 | 0.40% |
| 2 | 2.254 | 0.54% |
| 4 | 2.261 | 0.85% |
| 5 | 2.265 | 1.03% |
| 24 | 2.618 | 16.77% |

The tests performed on the samples show that the products according to the invention are 2 times less hygroscopic than the Adimix® precision powder. Indeed, the Adimix® precision product dissolves twice as quickly. This could partly explain the rapid release of the Adimix® precision product in the stomach, unlike the products resulting from the invention.

### Example 12: Relationship Between Process Viscosity and Morphology

During the conditions used for the preparation of the suspensions of sodium butyrate in the liquid fatty material of the processes according to the invention allowing obtaining granules of the invention, the viscosity of the suspension is assessed. It is compared with the morphology of the obtained granules and with the composition of the granules in FIG. 4.

The viscosity is analyzed by introducing an amount of 10 ml to fill the thermostated chamber of a Brookfield digital DV-E viscometer. The viscometer is configured with a coaxial cylinder in the chamber, using a S31 reference spindle. The viscosity measurement temperature is 85° C. The viscosity is determined for a rotational speed of the spindle of 10 rpm.

One could notice that at viscosity values lower than 3,000 mPa·s or 2,500 mPa·s of the suspensions of the process, granules with a spherical morphology are obtained.

### Example 13: Level of Free Butyric Acid by Extraction in the Organic Phase

The granules of the product of the invention PR1G1F, prepared without minerals in the matrix, are compared with the granules of the marketed product Adimix® precision. The granules have been crushed and have been extracted in an organic phase of hexane. Sodium butyrate is not soluble in hexane, it should remain in its solid form whereas butyric acid is miscible with hexane. Afterwards, the extracted solutions have been analyzed by CPG, phase chromatography, in order to determine the amount of butyric acid in the organic phase. It should be noted that there has been no visual observation of fatty acid dissolution in the organic phase.

Table 8 below reports the release rates for PR1G1F and Adimix® precision

TABLE 8
Free butyric acid level

| | Test 1 | Test 2 | Test 3 | Test 4 | Average |
|---|---|---|---|---|---|
| Adimix ®precision | 0.3% | 0.2% | 1.4% | 0.4% | 0.6% |
| PR1G1F | 0.0% | 0.0% | 0.0% | 0.0% | 0.0% |

These results indicate the presence of free butyric acid in the range of 0.6% in the Adimix® precision product, whereas no butyric acid content has been detected in the product PR1G1F of the invention.

### Example 14: Level of Esterification by Extraction in Aqueous Phase

The granules of the products of the invention PR1G1F and PCaR1G1F are compared with the marketed product Adimix® precision. The granules have been crushed and have been extracted in water. Then, the solutions have been analyzed by ion chromatography. The method does not identify whether the butyrate is in the acid (butyric) or basic (butyrate) form in the granule.

Table 9 reports the content of butyric acid or butyrate present in the extracted aqueous solution.

TABLE 9
Butyric acid or butyrate content

| | Test 1 | Test 2 | Average |
|---|---|---|---|
| Adimix ®precision | 21.8% | 23.0% | 22.4% |
| PCaR1G1F | 28.9% | 29.6% | 29.3% |
| PRIGF | 30.1% | 29.6% | 29.8% |

The products of the invention PR1G1F and PCaR1G1F have a sodium butyrate titer close to the initial nominal value of 30% unlike the Adimix® precision product.

The Adimix® precision product has a titer of 22.4% indicating that part of the butyrate initially introduced is not available in the form of butyric acid or butyrate. It is assumed that the presence of butyrate bound to other molecules could result from the esterification reaction with fatty acids or triglycerides. This assumption seems to be confirmed by the analysis reported in Example 15 below.

### Example 15: Identification of the Presence of Ester by Phase Change Analysis in Basic Medium

Part of the organic phase in hexane from Example 13 for the Adimix® precision product is sampled under stirring and an equivalent amount of 0.2 M sodium hydroxide. After stirring and centrifugation, the two organic and aqueous phases are analyzed. This phase change step in a basic medium should enable the sodium butyrate to dissolve in the basic aqueous phase and the butyric acid to pass from the organic phase to the aqueous phase in the form of butyrate. In particular, sodium hydroxide also allows saponifying butyrate if it is bound to the triglycerides or fatty acid in presence.

Table 10 below reports the extraction results of Adimix® precision during a phase change in a basic medium compared with the results obtained by extraction in water.

TABLE 10
Sodium butyrate or butyric acid content
after phase change in basic medium.

| Extraction | Adimix ®precision |
|---|---|
| Test 1 phase change | 27.6% |
| Test 2 phase change | 27.0% |
| Water extraction | 22.4% |

These results demonstrate that the phase change with sodium hydroxide allows saponifying and releasing butyrate in a bound form. The butyrate content is higher in the phase change (27.6%) than in the water extraction (22.4%) for Adimix® precision. This seems to confirm the presence of ester at least at 5.2% in total weight, i.e. a level of about 17% of butyrate will be in the form bound to triglycerides or fatty acids.

Conversely, a phase change extraction by adding water to the hexane phase of Example 13 for PR1G1F, after analysis of the solution reveals a butyrate content of 29%, similar to the content by water extraction (29.8%). The results in Table 11 confirm that the product of the invention PR1GF1 has only a little amount of butyrate in a bound form.

TABLE 11
Sodium butyrate or butyric acid content after phase change.

| Extraction | PR1GF1 |
|---|---|
| Test 1 phase change | 27.7% |
| Test 2 phase change | 30.30% |
| Water extraction | 29.8% |

1. A granule comprising:
sodium butyrate particles,
a fatty material matrix comprising fatty acids encapsulating the sodium butyrate particles,
the granule preserving its morphology following gastric and enteric digestion simulation in vitro tests,
the granule featuring a gastric resistance conferring protection on the sodium butyrate particles in the stomach and featuring a sustained release of the sodium butyrate particles in the intestinal tract,
wherein the gastric protection rate TRC1 of sodium butyrate is higher than or equal to 50%, the gastric protection rate TRC1 being the relative amount of sodium butyrate protected from gastric digestion after the incubation in vitro test in the gastric environment adapted according to Boisen's method
and/or
wherein:
the enteric release rate TRC2 of sodium butyrate in the small intestine is higher than or equal to 25%, the enteric release rate TRC2 being the relative amount of sodium butyrate released and dissolved in solution after the incubation in vitro test in the enteric environment of the small intestine, adapted according to Boisen's method, and
the enteric release rate TRC3 of sodium butyrate in the large intestine is higher than or equal to 50% respectively, the enteric release rate TRC3 being the relative amount of sodium butyrate released and dissolved in solution after the incubation in vitro test in the enteric environment of the large intestine, adapted according to Boisen's method.

2. The granule according to claim 1, wherein the butyric acid level is lower than 5% of the total amount of sodium butyrate.

3. The granule according to claim 1, wherein the fatty material comprises long-chain fatty acids containing more than 12 carbon atoms, the C16 and C18 fatty acids being at a content higher than or equal to 70% of the total weight of the fatty material, the C16:C18 weight ratio of the fatty acids being from 0.7 to 1.7.

4. The granule according to claim 1, wherein the morphology is spherical.

5. The granule according to claim 1, wherein the matrix comprises one mineral or several minerals
at a mineral percentage of 2% to 10% of the total weight of the granule,
the mineral being selected from among calcium carbonate, tricalcium phosphate, calcium sulfate, calcium silicate, magnesium sulfate, magnesium carbonate, aluminum phosphate, cobalt carbonate, zinc carbonate and mixtures thereof.

6. The granule according to claim 1, wherein the granule comprises:
from 40% to 80% by weight of fatty material,
and from 20% to 60% by weight of sodium butyrate,
and wherein the fatty material is selected from the group consisting of hydrogenated palm oil, hydrogenated sunflower oil, hydrogenated rapeseed oil, beeswax, candelilla wax, carnauba wax, palm stearin, stearic acid or a mixture thereof.

7. A population of granules comprising granules according to claim 1,
wherein the particle size of the granules varies from 200 μm to 1.5 mm, and wherein the size of the sodium butyrate particles varies from 50 to 1,200 μm;
or wherein the SPAN value of the granules is lower than or equal to 1; the SPAN value being calculated according to the following formula:
$$SPAN = \frac{D ( 90 \% ) - D ( 10 \% )}{D ( 50 \% )}$$
wherein D(90%), D(50%) and D(10%) represent the diameters for which respectively 90%, 50% and 10% of the population of granules has a diameter smaller than this value,
or wherein the population of granules forming a powder has a flow index of 4 to 7 [Flodex™ index],
or wherein the moisture pick-up value is from 3 to 10%.

8. A population of granules comprising granules according to claim 1, wherein at least 90% by weight of the granules has a spherical morphology.

9. A method for preparing a population of granules comprising
sodium butyrate particles,
a fatty material matrix comprising fatty acids, encapsulating the sodium butyrate particles,
the granule preserving its morphology following gastric and enteric digestion simulation in vitro tests,
the granule featuring a gastric resistance conferring protection on the sodium butyrate particles in the stomach and featuring a sustained release of sodium butyrate particles in the intestinal tract,
the method comprising:
a step of preparing a mixture of sodium butyrate particles in the solid state in the liquid of the fatty material in the molten state to obtain a suspension
a step of forming granules from the suspension, possibly subsequently solidified in the form of a dispersion of particles in the crystallized fatty material,
wherein the viscosity of the suspension consisting of the mixture of the sodium butyrate particles in the molten liquid of the fatty material before the aforementioned step of forming the granules is lower than 8,000 mPa·s.

10. The method according to claim 9,
comprising:
a step of preparing a mixture of a powder of sodium butyrate particles in a fatty material in the molten state in liquid form to obtain a suspension,
a step of forming drops from the suspension,
a step of cooling the drops into granules enabling the solidification of the fatty material forming a matrix encapsulating the sodium butyrate particles;
or comprising:
a step of preparing a mixture of a powder of sodium butyrate particles in a fatty material in the molten state in liquid form, by introducing the powder into the fatty material in the molten state in liquid form by appropriate means to obtain a suspension,
a step of forming drops by spraying the aforementioned suspension,
a step of cooling the drops in a cooling chamber at a temperature from −20° C. to 30° C.;
or comprising:
a step of preparing a mixture of a powder of sodium butyrate particles and of the fatty material in powder form, followed by raising the temperature of the mixture to melt the fatty material containing the sodium butyrate particles, to obtain a suspension
a step of forming the drops of the aforementioned suspension,
a step of cooling the drops in a cooling chamber at a temperature from −20° C. to 30° C.;
or comprising:
a step of preparing a mixture of a powder of sodium butyrate particles and of the fatty material in powder form, followed by raising the temperature of the mixture to melt the fatty material containing the sodium butyrate particles to obtain a suspension,
a step of cooling the suspension to obtain a dispersion of particles in the crystallized fatty material,
a step of forming granules from the dispersion.

11. The method according to claim 9, wherein the fatty material comprises long-chain fatty acids containing 16 and 18 carbon atoms at a content higher than or equal to 70% of the total weight of the fatty material the C16:C18 weight ratio of the fatty acids is from 0.7 to 1.7.

12. The method according to claim 9, wherein the fatty material matrix comprises at least one mineral in an amount of 2% to 10% of the total weight of the granule.

13. A population of granules which can be obtained according to the method according to claim 9.

14. An animal and human feed composition comprising granules according to claim 1 or a population of granules
wherein the particle size of the granules varies from 200 μm to 1.5 mm, and wherein the size of the sodium butyrate particles varies from 50 to 1,200 μm;
or wherein the SPAN value of the granules is lower than or equal to 1; the SPAN value being calculated according to the following formula:
$$SPAN = \frac{D ( 90 \% ) - D ( 10 \% )}{D ( 50 \% )}$$
wherein D(90%), D(50%) and D(10%) represent the diameters for which respectively 90%, 50% and 10% of the population of granules has a diameter smaller than this value,
or wherein the population of granules forming a powder has a flow index of 4 to 7 [Flodex™ index],
or wherein the moisture pick-up value is from 3 to 10%.
